(19) United States
(12) Patent Application Publication
(10) Pub. No.: US 2023/0147203 A1
(43) Pub. Date: May 11, 2023

(54) CONNECTING ELEMENT FOR A PATIENT INTERFACE

(71) Applicant: Loewenstein Medical Technology S.A., Luxembourg (LU)

(72) Inventors: Jennifer Gruber, Hamburg (DE); Sarah Kamerling, Geeste (DE)

(21) Appl. No.: 18/053,004

(22) Filed: Nov. 7, 2022

(30) Foreign Application Priority Data
Nov. 10, 2021 (DE) 102021005565.9

(51) Int. Cl. A62B 18/08

(52) U.S. Cl. CPC A62B 18/084

(57) ABSTRACT

A connecting element for a patient interface, which connecting element comprises at least one strap which is configured for connection to the patient interface and comprises two straps and at least one behind-the-ear element.

### CROSS-REFERENCE TO RELATED APPLICATIONS

The present application claims priority under 35 U.S.C. § 119 of German Patent Application No. 102021005565.9, filed Nov. 10, 2021, the entire disclosure of which is expressly incorporated by reference herein.

### BACKGROUND OF THE INVENTION

### 1. Field of the Invention

The invention relates to a connecting element for a patient interface.

### 2. Discussion of Background Information

Patient interfaces, or respiratory masks, are used for ventilation, to support breathing or as a protective mask against aerosols of solid or liquid particles. Respiratory masks for ventilation or respiratory support form the interface between a user or patient and a ventilator and must be fixed to the head of the user by means of a connecting element. Both the respiratory masks and the connecting elements must satisfy high demands in terms of stability and safety and at the same time offer sufficient comfort and simple handling for the user. A distinction is made between respiratory masks with forehead supports and respiratory masks without forehead supports.

Respiratory masks without forehead supports conventionally have stiffening devices in the connecting elements in order to ensure adequate stability and a good fit of the respiratory mask. The stiffened region is generally cushioned toward the face of the user in order to increase wear comfort. The connecting elements are conventionally of complex construction and are sometimes not easy to use.

There is thus a need for connecting elements which are simple to handle and ensure a stable, secure and comfortable fit of the respiratory mask.

### SUMMARY OF THE INVENTION

The invention provides a connecting element for a patient interface, wherein the connecting element comprises at least one strap which is configured for connection to the patient interface. According to the invention, the connecting element comprises two straps and at least one behind-the-ear element.

In some embodiments, the connecting element is characterized in that the at least one behind-the-ear element can be coupled reversibly and/or irreversibly to the straps.

In some embodiments, the connecting element is characterized in that the straps comprise a beginning and an end, wherein a coupling system for coupling the strap to the patient interface is disposed and configured adjacent to the beginning.

In some embodiments, the connecting element is characterized in that an end portion to which the behind-the-ear element can be coupled is arranged adjacent to the end.

In some embodiments, the connecting element is characterized in that the straps are produced at least from a stiffening and at the same time flexible material and/or composite material selected from plastics materials, metals, silicones and foams.

In some embodiments, the connecting element is characterized in that the straps are manufactured from polypropylene.

In some embodiments, the connecting element is characterized in that the straps have a length and a width, wherein the ratio of the length to the width is at least about 10:1, preferably at least about 20:1, particularly preferably at least about 25:1.

In some embodiments, the connecting element is characterized in that the straps additionally have a cushioning material with elastic and/or viscoelastic properties selected from foams, cotton wool, woven fabric, cotton, wool, rubber, neoprenes and gels.

In some embodiments, the connecting element is characterized in that the behind-the-ear element is manufactured from an elastic material selected from silicones, thermoplastic elastomers, neoprenes and textiles.

In some embodiments, the connecting element is characterized in that the behind-the-ear element is manufactured from silicone.

In some embodiments, the connecting element is characterized in that the behind-the-ear element comprises at least one retaining element which can be coupled to the end portion of the strap.

In some embodiments, the connecting element is characterized in that the retaining element is in the form of a hollow body with a lumen into which the end portion of the strap is placed for coupling at at least one position.

In some embodiments, the connecting element is characterized in that the retaining element is displaceably coupled at any desired positions of the end portion and a length adjustment of the connecting element is thus provided.

In some embodiments, the connecting element is characterized in that the end portions comprise at least one anchor element for anchoring the coupling of the retaining element to the straps.

In some embodiments, the connecting element is characterized in that the end portions alternatively or additionally comprise one or more fixing elements for fixing the coupling of the behind-the-ear element to the straps.

In some embodiments, the connecting element is characterized in that the fixing elements are formed by one or more thickened portions and/or indentations of the end portion of the straps.

In some embodiments, the connecting element is characterized in that the behind-the-ear element has counterpart elements corresponding to the fixing elements for assisting with fixing of the behind-the-ear element to the straps.

In some embodiments, the connecting element is characterized in that the fixing elements are in the form of buttons, mushroom buttons, hooks, bars, studs or Velcro tapes with hooks and/or loops, and the counterpart elements are in the form of buttonholes, holes, eyelets, loops or Velcro tapes with hooks and/or loops, and the fixing elements can be connected to the counterpart elements such that the behind-the-ear element is fixed to the straps.

In some embodiments, the connecting element is characterized in that the fixing of the behind-the-ear element to the straps permits a stepwise length adjustment of the connecting element.

In some embodiments, the connecting element is characterized in that the connecting element additionally comprises a head band which can be coupled to the straps.

In some embodiments, the connecting element is characterized in that the straps and the behind-the-ear element are in one-piece or two-piece form.

In some embodiments, the connecting element is characterized in that the behind-the-ear element has a length and a width, wherein the ratio of the length to the width of the behind-the-ear element is at least about 1:1, preferably at least about 5:1.

In some embodiments, the connecting element is characterized in that the connecting element comprises two straps and one behind-the-ear element with two retaining elements, wherein one retaining element is coupled to one strap and the second retaining element is coupled to the second strap.

In some embodiments, the connecting element is characterized in that the one behind-the-ear element runs across the back of the head of a user or patient in a use state.

In some embodiments, the connecting element is characterized in that the ratio of the length to the width of the behind-the-ear element is at least about 5:1, preferably at least about 20:1, particularly preferably at least about 30:1.

In some embodiments, the connecting element is characterized in that the behind-the-ear element is in itself adjustable in length.

In some embodiments, the connecting element is characterized in that the behind-the-ear element comprises at least one eyelet which is configured and adapted to permit a length adjustment.

In some embodiments, the connecting element is characterized in that the connecting element comprises two straps and two behind-the-ear elements each with a retaining element, wherein a behind-the-ear element is coupled to each of the two straps.

In some embodiments, the connecting element is characterized in that the ratio of the length to the width of the behind-the-ear elements is in each case less than about 20:1, preferably less than about 10:1, particularly preferably less than about 5:1.

In some embodiments, the connecting element is characterized in that the behind-the-ear elements run behind the ear of a user or patient in a use state.

The object is further achieved by a system as claimed in claim 19. The system for ventilation and/or respiratory support comprises at least a patient interface, the connecting element according to the invention for connection to the patient interface, a ventilator, at least one line, wherein the patient interface is coupled with the connecting element and is connected by way of the connecting element to the head of a patient, wherein the patient interface and the ventilator are connected together in a gas-conducting manner by way of the line.

### BRIEF DESCRIPTION OF THE DRAWINGS

Exemplary embodiments of the connecting element according to the invention are shown in the figures, in which:

FIG. 1 shows, by way of example, an overview of the system with an exemplary embodiment of the connecting element according to the invention which is connected to a patient interface.

FIG. 2 is a perspective top view of a part-region of the connecting element.

FIG. 3 is a schematic side view of an embodiment of the connecting element in a use state on the head of a user.

FIG. 4 and FIG. 5 are side views of the strap.

FIG. 6A shows the connecting element according to the invention according to FIG. 3 in a view from above and FIG. 6B is a view from above of the connecting element in a use state on the head of a user.

FIGS. 7 to 9 are different views of the connecting element according to the invention from FIG. 3, wherein

FIG. 7 is a perspective view of the connecting element, FIG. 8A and FIG. 8B are views of the connecting element from the side, and FIG. 9 is a view of the connecting element from the front.

FIGS. 10A-C show a part-region of the strap in different embodiments.

FIGS. 11A, 11B, 12A, 12B, 12C, 13A, 13B, 13C, 13D, 14A, 14B, 14C and 14D show different embodiments of a behind-the-ear element of the connecting element according to the invention from FIG. 3.

FIG. 15 is a schematic side view of an alternative embodiment of the connecting element according to the invention in a use state on the head of a user.

FIGS. 16, 17, 18A and 18B are different views of the connecting element according to the invention from FIG. 15, wherein FIG. 16 is a view of the connecting element from the side,

FIG. 17 is a perspective view of the connecting element, FIG. 18A is a view of the connecting element from the front, and FIG. 18B is a view of the connecting element from above.

FIGS. 19A-C show an exemplary embodiment of the behind-the-ear element of the connecting element according to the invention from FIG. 15, wherein FIG. 19A is a view from the side, FIG. 19 B is a view from the front, and FIG. 19 C is a perspective view.

FIGS. 20A-D show alternative exemplary embodiments of the behind-the-ear element of the connecting element according to the invention from FIG. 15 from the side.

The coordinate systems shown in some figures serve to illustrate the orientation of the views. The x-, y- and z-axes—unless described otherwise—in each figure describe the same direction. A direction denoted with an x-axis of one figure thus generally corresponds to the direction of the x-axis of the other figures.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

In the following exemplary embodiments, a connecting element 10 according to the invention is shown.

The connecting element 10 according to the invention is suitable, for example, for use in a system 100 for ventilation and/or respiratory support. The system 100 comprises a patient interface 90 and a ventilator 70, which are connected together in a gas-conducting manner by way of a line 80. The patient interface 90 is connected by way of the connecting element 10 to the head 95 of a patient.

The connecting element 10 is in a use state when the connecting element 10 is applied at least partially at least in some regions to the head 95 of a patient and/or user.

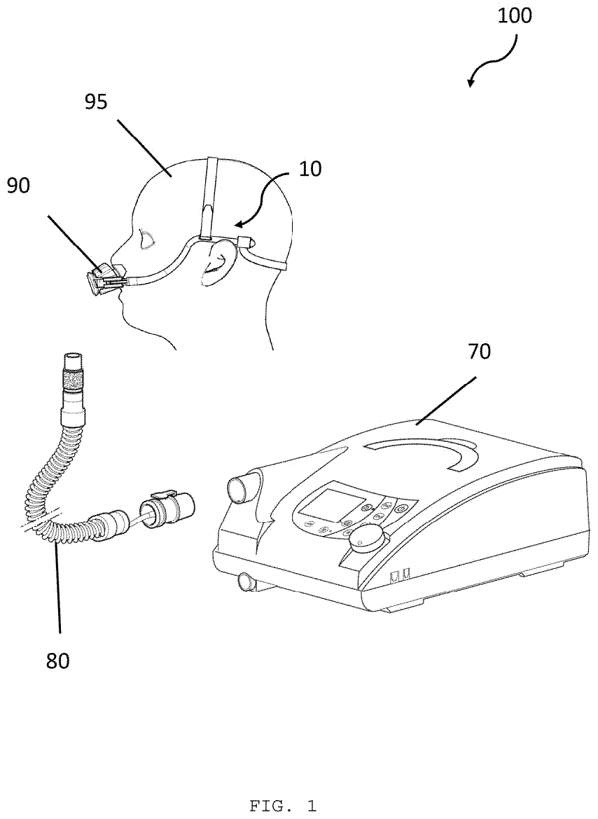

FIG. 1 shows, by way of example, an overview of the system 100 with an exemplary embodiment of the connecting element 10 according to the invention, which is connected to a patient interface 90. The patient interface 90 can be connected in a gas-conducting manner by way of a line 80 to a ventilator 70. In FIG. 1, the patient interface 90, line 80 and ventilator 70 are shown not connected. A gas-conducting connection can be established by connecting the line 80 by way of connectors, for example, at one end to the patient interface 90 and at a further end to the ventilator 70 (not shown).

The patient interface 90 is to be understood as being any peripheral device which is configured for interaction with a living being. In particular, the patient interface 90 is configured for therapeutic and/or diagnostic purposes in conjunction with the ventilator 70. The patient interface 90 can be in the form of a respiratory mask. Patient interface 90 and respiratory mask are used herein as synonyms.

The patient interface 90 can be in the form of a nasal mask and can enclose the nose completely or partially. The patient interface 90 can also be in the form of a nasal pillow mask. Nasal pillow masks are used, for example, in CPAP or bilevel therapy. The patient interface 90 can also be in the form of a nasal cannula or oxygen nasal cannula. Nasal cannulas are used, for example, in high-flow therapy. Nasal cannulas can have nasal prongs 92, which can be inserted at least partially into the nostrils. The patient interface 90 can also be in the form of a full face mask or total face mask, that is to say can enclose the nose and the mouth. Tracheal tubes or cannulas can also be used as the patient interface 90. The connecting element 10 according to the invention is suitable in particular for nasal pillow masks and/or for high-flow interfaces.

The patient interface 90 can be applied or fastened to the head 95 of a user or patient by way of a connecting element 10 according to the invention. User and patient are used synonymously herein. When the term user or patient is mentioned, any individual who uses a respiratory mask 90 with a connecting element 10 is meant.

A ventilator 70 is to be understood as being all devices which support a user or patient with natural breathing and/or undertake ventilation of a user or patient and/or serve for respiratory therapy and/or otherwise act on the breathing of a user or patient. They include, for example but not exclusively, respiratory therapy devices, CPAP, APAP and bilevel devices, high-flow therapy devices, anesthesia machines, clinical, out-of-hospital or emergency ventilators, oxygen O₂-delivering devices, diagnostic systems and cough-assist machines.

The patient interface 90 and the ventilator 70 are preferably connected together by way of at least one line 80. The line 80 is in the form of a gas line which connects the individual components of the system 100 together in a gas-conducting manner. The line 80 is preferably flexible and/or rotatable. The line 80 can be in the form of, for example, an elastic tube and/or hose and/or hose system.

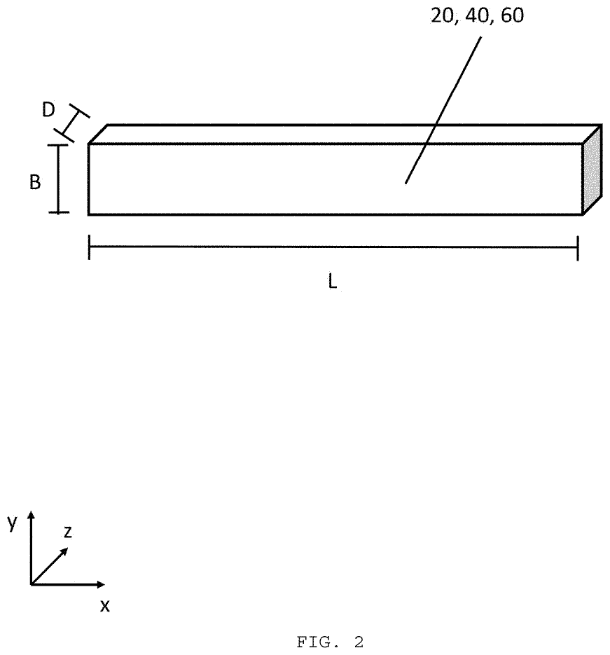

FIG. 2 is a perspective top view of a part-region of the connecting element 10. The connecting element 10 and portions of the connecting element 20, 40, 60 have a width B, a thickness D and a length L. The width B is usually greater than the thickness D. The length L is usually greater than the width B. In a use state, that is to say when the connecting element 10 is applied at least partially to the head 95 (not shown here) of the user, the connecting element 10 faces the head 95 of the user at least in some regions on one side along the width B and along the length L.

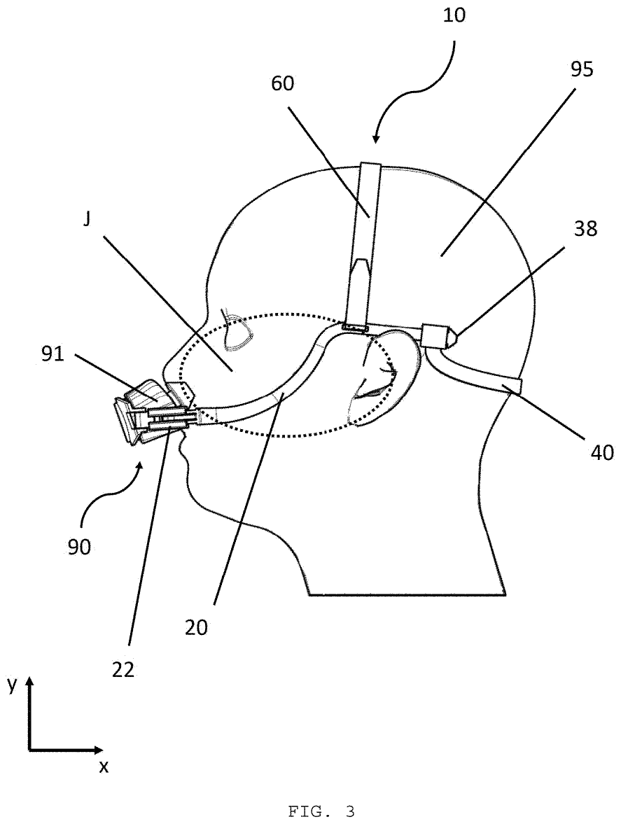

FIG. 3 is a schematic side view of an embodiment of the connecting element 10 in a use state on the head 95 of a user. The connecting element 10 comprises at least one strap 20 and at least one behind-the-ear element 40. In addition, the connecting element can comprise at least one head band 60.

It is apparent from FIG. 3 that the connecting element 10, in a use state, is arranged on the head 95 of a patient such that the strap 20 is arranged at least in part between the nose and the ear. The strap 20 is thereby located at least in part in a cheekbone region J.

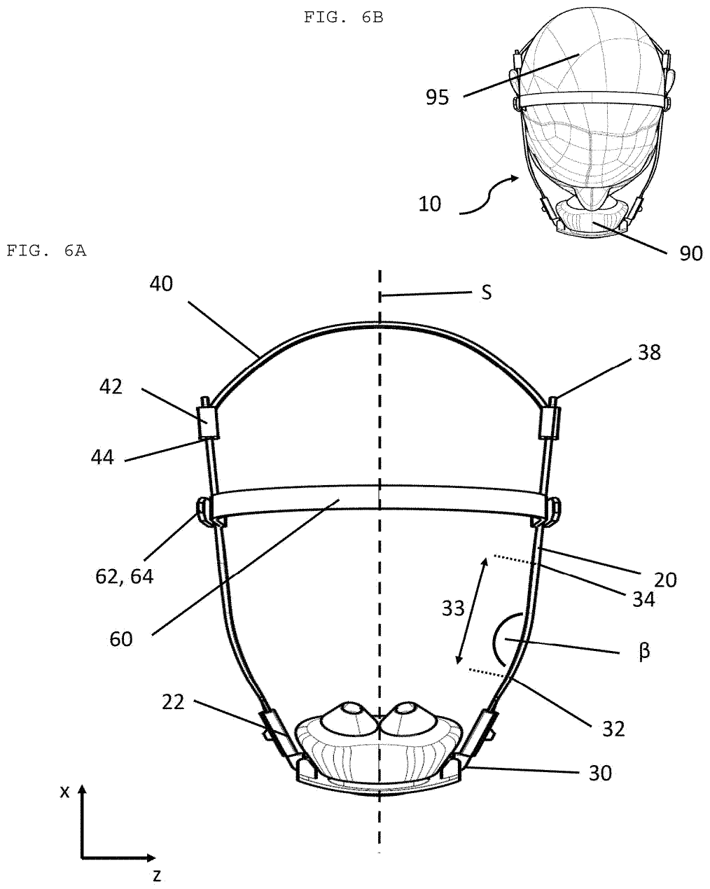

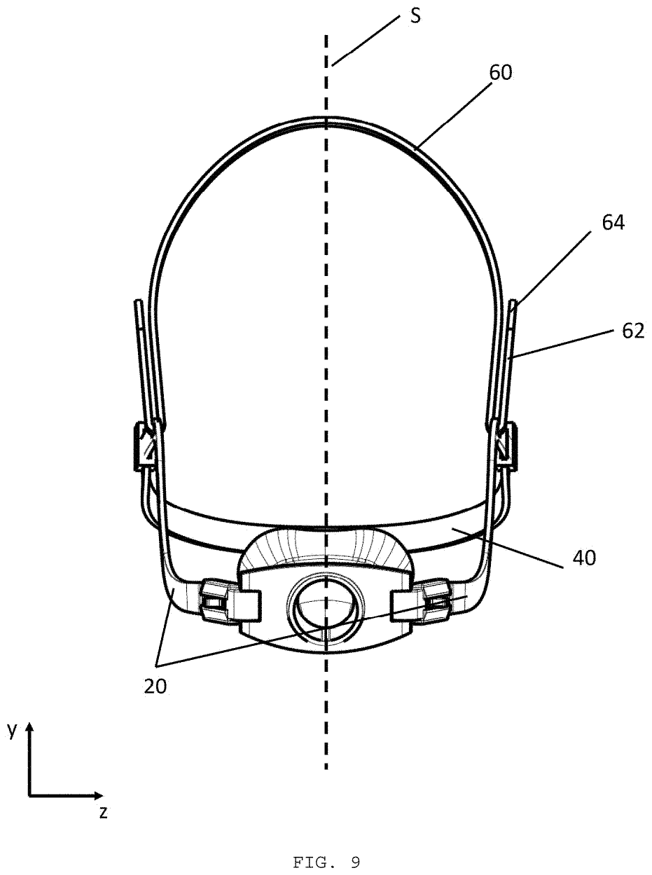

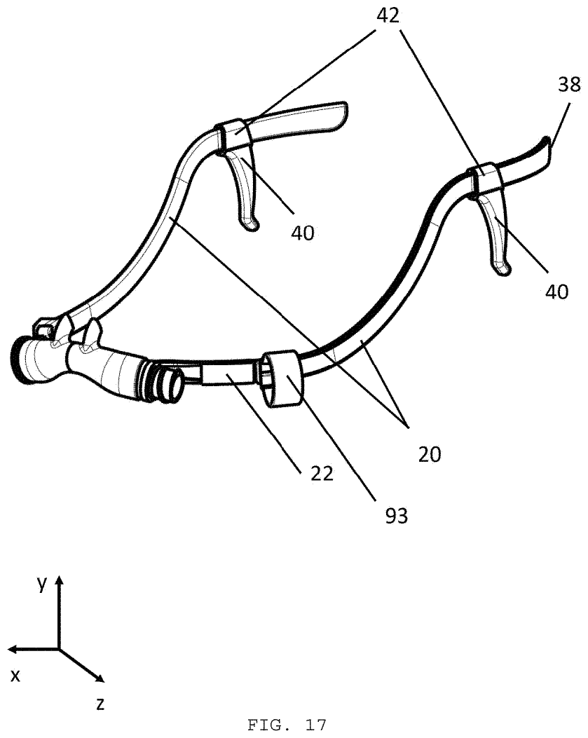

The connecting element 10 can be fundamentally mirror-symmetrical. The connecting element 10 can therefore have a notional plane of symmetry S (see FIGS. 6A, 9) which runs parallel to the plane spanned by the x- and y-axes (see for example FIG. 3). Fundamentally mirror-symmetrical means that perfect symmetry is not generally present and the mirror images can differ from one another in one or more details. The connecting element 10 can additionally have one or more additional elements which, irrespective of the fundamental symmetry, can additionally be arranged on one side of the connecting element 10. An example which may be mentioned here is a hose support 93, which can be arranged on only one of the two straps 20 (see for example FIG. 17). In the following text, only one of the mirror-symmetrical halves of the connecting element 10 will generally be described in each case.

The connecting element 10 comprises at least one strap 20. The connecting element 10 generally comprises two straps 20. In a use state, the straps 20 are located on the one hand over parts of the left side of the face and on the other hand over parts of the right side of the face (not shown). The connecting element 10 can also comprise more than two straps 20.

The strap 20 comprises a coupling system 22. By way of the coupling system 22, the connecting element 10 can be coupled to the patient interface 90. Coupling can take place reversibly or irreversibly. The coupling system 22 can comprise a mechanism for length adjustment. By way of the coupling system 22, the length L of the connecting element 10 can thus be adapted. The coupling can be movable or unmovable.

The patient interface 90 can be in the form of a nasal pillow mask. Nasal pillow masks can be used, for example, for CPAP or bilevel therapies. For this purpose, the nasal pillow masks cover the nostrils and/or the region around the nostrils in a gas-tight manner with a nasal pillow 91. The nasal pillow 91 can be inserted at least partially into the nostrils.

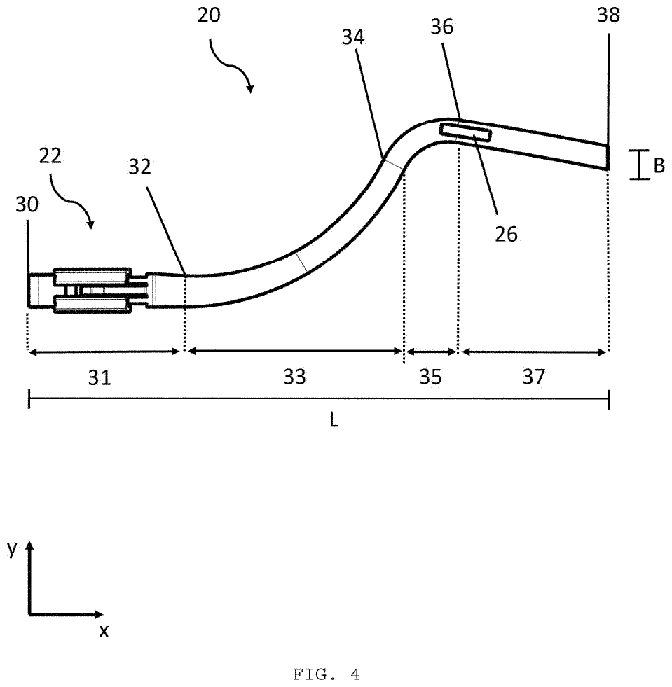

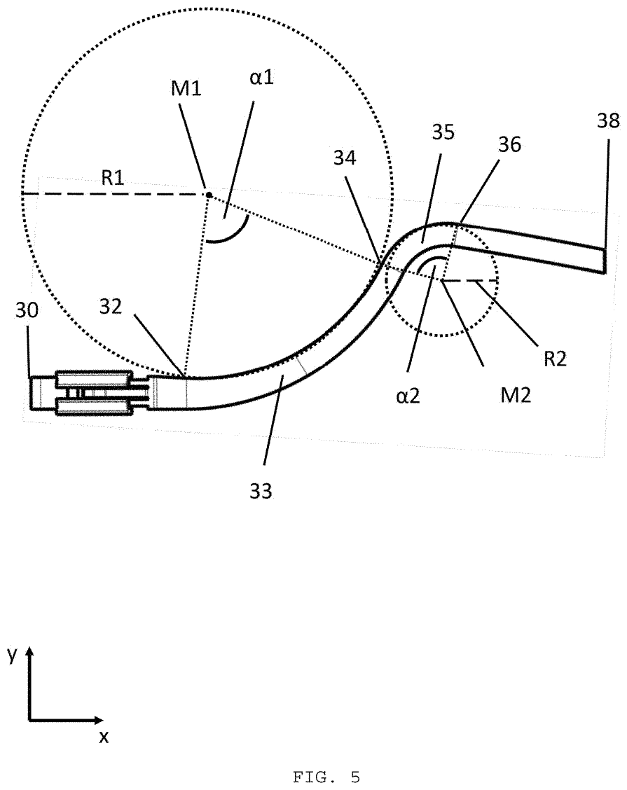

FIG. 4 and FIG. 5 are side views of the strap 20. The strap 20 comprises a beginning 30 and an end 38. The beginning 30 and the end 38 lie at opposite ends of the strap 20 along the length L of the strap 20. The strap 20 ends on one side at the beginning 30 and on the other side at the end 38. In a use state, the beginning 30 can be arranged adjacent to the patient interface 90 and the end 38 can be arranged on the opposite side. The end 38 can be arranged on and/or behind the ear in a use state (see FIG. 3).

The strap 20 extends along the length L from the beginning 30 to the end 38. The length L of the strap 20 is substantially greater than the width B of the strap 20. The ratio of the length L to the width B of the strap 20 can be in a range from about 10:1 to about 40:1. For example, the ratio of the length L to the width B of the strap 20 is at least about 10:1. In some embodiments, the ratio of the length L to the width B of the strap 20 is at least about 20:1. In a preferred embodiment, the ratio of the length L to the width B of the strap 20 is, for example, about 25:1 or more. The strap 20 has a particularly elongate, narrow shape.

The width B of the strap 20 can be constant or variable. In one exemplary embodiment, the width B of the strap 20 can be substantially constant and can increase or decrease in the width B toward the beginning 30 and/or toward the end 38.

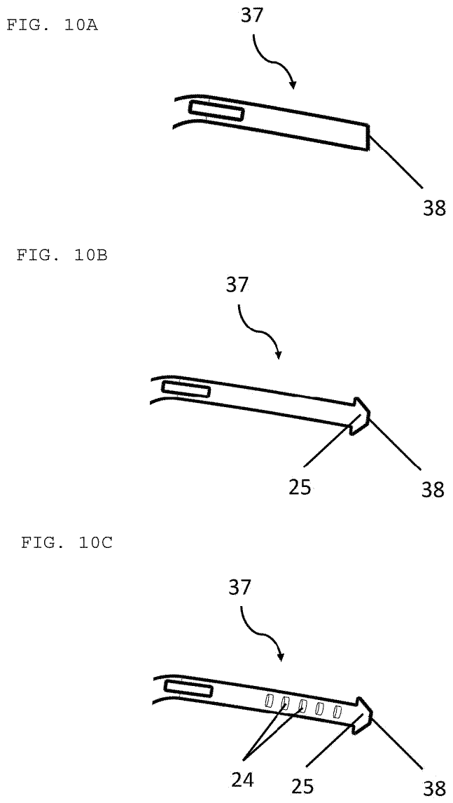

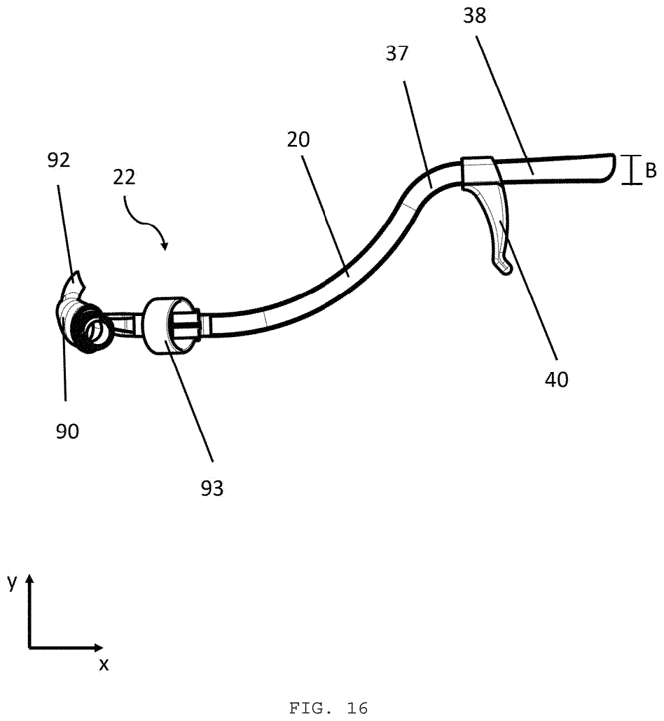

In a preferred embodiment, the width B increases toward the end 38 (see for example FIG. 10B, FIG. 16). The width B can increase or decrease toward the beginning 30. In a preferred embodiment, the width B increases toward the beginning 30 (FIG. 4).

The width B of the strap 20 can be in a range from about 1 mm to about 20 mm. Preferably, the width B of the strap 20 is from about 3 mm to about 10 mm. In a concrete exemplary embodiment, the width B of the strap 20 is about 7 mm.

The length L of the strap 20 can be in a range from about 50 mm to about 250 mm. Preferably, the length L of the strap 20 is from about 150 to about 220 mm. In a concrete exemplary embodiment, the length L of the strap 20 is, for example, about 200 mm.

It is apparent from FIG. 4 that the strap 20 can be straight and/or bent at least in part. FIG. 4 and FIG. 5 show that the strap 20 can comprise a plurality of portions 31, 33, 35, 37 which can have different forms. The strap 20 can comprise a plurality of inflection points 30, 32, 34, 36, 38 between which portions 31, 33, 35, 37 are arranged.

The beginning 30 of the strap 20 can lie at the point of intersection of the x- and y-axes. The beginning 30 defines a first outer end of the strap 20, namely the end that is adjacent to the patient interface 90 and to the coupling system 22 in a use state. The end 38 of the strap 20 defines a second outer end of the strap 20, namely the end that is arranged on and/or behind the ear in a use state. The strap 20 can run between the beginning 30 and the end 38 straight and/or bent or curved in the x/y coordinate system. The strap 20 extends in the x/y coordinate system preferably such that the end 38 lies above the beginning 30 in the y-direction.

One or more inflection points 32, 34, 36 can lie between the beginning 30 and the end 38. In the concrete exemplary embodiment shown in FIGS. 4/5, three inflection points 32, 34, 36, for example, lie between the beginning 30 and the end 38. In alternative embodiments, fewer than three or more than three inflection points can also be formed.

One or more portions 31, 33, 35, 37 can lie between the beginning 30 and the inflection points 32, 34, 36 and the end 38. In the concrete exemplary embodiment shown in FIGS. 4/5, four portions 31, 33, 35, 37, for example, lie between the beginning 30 and the end 38. In alternative embodiments, fewer than four or more than four portions can also be formed. The portions 31, 33, 35, 37 can be straight and/or bent or curved.

A portion 31 of the strap 20 can be arranged between the beginning 30 and the inflection point 32. Portion 31 denotes the strap portion that lies between the beginning 30 and the inflection point 32. The portion 31 can be straight or bent or curved. In the concrete exemplary embodiment according to FIG. 4, the portion 31 is substantially straight in the direction of the x-axis. The coupling system 22 can be arranged in the portion 31. A mechanism for length adjustment can be arranged in the portion 31.

A further portion 33 of the strap 20 can be arranged between the inflection point 32 and the inflection point 34. Portion 33 denotes the strap portion that lies between the inflection point 32 and the inflection point 34. The portion 33 can be straight or bent or curved. In the concrete exemplary embodiment according to FIGS. 4/5, the portion 33 is bent in the x/y coordinate system. The inflection point 34 lies above the inflection point 32 in the y-direction.

A further portion 35 of the strap 20 can be arranged between the inflection point 34 and the inflection point 36. Portion 35 denotes the strap portion that lies between the inflection point 34 and the inflection point 36. The portion 35 can be straight or bent or curved. In the concrete exemplary embodiment according to FIGS. 4/5, the portion 35 is bent in the x/y coordinate system. The inflection point 36 lies above the inflection point 34 in the y-direction.

FIG. 5 clearly shows the bend of the portions 33 and 35 of the concrete exemplary embodiment according to FIG. 4. Portion 33 runs in a bent manner between the inflection points 32 and 34. The bend corresponds to an arc of a notional circle or oval with a radius R1. The radius R1 can be in a range between 60 mm and 75 mm, for example. If the inflection points 32 and 34 are joined to a center M1 of the circle or oval, a sector with a central angle α1 is obtained. The central angle α1 can be in a range of from about 30° to about 80°. For example, the central angle α1 is in a range from about 60° to about 70°.

Portion 35 runs in a bent manner between the inflection points 34 and 36. The bend corresponds to an arc of a notional circle or oval with a radius R2. The radius R2 can be in a range from about 10 mm to about 20 mm, for example. If the inflection points 34 and 36 are joined to a center M2 of the circle or oval, a sector with a central angle α2 is obtained. The central angle α2 can be in a range from about 30° to about 80°. For example, the central angle α2 is in a range from about 60° to about 75°.

The central angles α1 and α2 can be of the same size or can differ from one another in terms of their size.

The bend or curve of the portion 35 can, for example, be opposite to the bend or curve of the portion 33. Thus, for example, a convexly bent strap portion 35 can adjoin a concavely bent strap portion 33 and vice versa.

In alternative embodiments, the strap 20, instead of being formed of the arcuate curves shown in FIGS. 4/5, can also be formed of straight portions 31, 33, 35, 37 which are bent relative to one another such that the end 38 lies above the beginning 30 in the y-direction (not shown).

An end portion 37 of the strap 20 is arranged between the inflection point 36 and the end 38. End portion 37 can denote the strap portion that lies between the inflection point 36 and the end 38. The end portion 37 is adjacent to the end 38 and denotes the last portion of the strap 20.

The end portion 37 can be straight and/or bent or curved. In the concrete exemplary embodiment according to FIG. 4, the end portion 37 is substantially straight in the direction of the x-axis.

In some embodiments, the end portion 37 can be substantially longer than is shown in FIGS. 4/5. The length L of the end portion 37 can account for up to about 80% of the total length L of the strap 20. Preferably, the length L of the end portion 37 accounts for less than about 50% of the total length L of the strap 20. In a concrete exemplary embodiment, the length L of the end portion 37 accounts for about 20% to about 25% of the total length L of the strap 20. A short end portion 37 has a positive effect on the wear comfort for the user, for example when the user puts his head down when lying down, for example when sleeping.

In some embodiments, it is conceivable that an end portion 37 of elongate form can be straight and curved or bent. It is conceivable, for example, that the end portion 37 of the strap 20 is curved at least in part such that it passes at least partially around the ear in a use state (not shown).

In the concrete exemplary embodiment shown in FIG. 4/5, there are four portions 31, 33, 35, 37, for example, between the beginning 30 and the end 38. In alternative embodiments, fewer than four or more than four portions can also be formed.

The end portion 37 is adapted and configured, at least in part, to enter into a coupling with the behind-the-ear element 40. By way of the end portion 37, the behind-the-ear element 40 can be connected to the strap 20.

In some embodiments, the strap 20 can additionally comprise at least one coupling element 26. The coupling element 26 can, for example and preferably, be in the form of an aperture in the strap 20. The coupling element 26 can also be in the form of an additional element, for example in the form of a loop or eyelet, which is molded onto the strap 20 (not shown).

The at least one coupling element 26 is adapted and configured to couple the head band 60 as an additional connecting element to the strap 20. The coupling element 26 can be arranged at all the portions 31, 33, 35, 37 of the strap 20. Preferably, the coupling element 26 is arranged in portion 35 and/or in the end portion 37.

In a concrete exemplary embodiment, the coupling element 26 can be formed by a cutout in the material of the strap 20 (see FIG. 4). The head band 60 can be guided through the coupling element 26. For this purpose, the coupling element 26 preferably has a complementary shape to the head band 60. For example, the coupling element 26 is minimally wider than the head band 60 is wide and minimally higher than the head band 60 is thick.

The strap 20 is preferably formed at least of a solid, stiffening material which nevertheless has a certain flexibility. The straps 20 should have a certain stiffness while at the same time having flexibility so as to impart sufficient stability to the connecting element 10 as a whole and nevertheless permit flexible contact with the head 95.

In a simple embodiment, the strap 20 can be manufactured from only one material, which forms a layer. In some embodiments, it is also possible that the strap 20 is composed of a plurality of layers (e.g., two or three layers).

The strap 20 can be formed of plastics material, metal, silicone, foam and/or a composite material. The base material of the strap 20 can be rigid plastics material, for example. For example, the strap 20 is manufactured from polypropylene.

The strap 20 can have additional reinforcing and/or flexible and/or deformable elements in a base material, at least in some regions. For example, there can additionally be arranged in a base material of plastics material, for example, wire, inorganic and/or organic reinforcing fibers, short and/or long fibers, rods, strips, woven fabrics, knitted fabrics, meshes and/or structural mats.

In some embodiments, the strap 20 can be in the form of a deformable element which can be adapted individually to the shape of the head. For example, the strap 20 can be formed of at least one wire of a wrought alloy, for example of a wrought aluminum alloy. Other materials with deformable properties are also conceivable, for example plastics materials such as thermoplastics or the like. In an alternative embodiment, the strap 20 can be formed of a composite material, preferably of a rigid plastics material and silicone and/or of a rigid plastics material and a thermoplastic elastomer (TPE).

The strap 20 is not limited to these embodiments. Any materials which have reinforcing and/or stiffening and/or flexible properties are conceivable. In a simple embodiment, the strap 20 can be manufactured from only one of the above-mentioned materials or composite materials.

In some embodiments, the strap 20 can additionally be surrounded at least in some regions by a textile. The textile can increase the wear comfort of the strap 20. Conceivable textiles can be cotton, silk and/or of synthetic fibers such as, for example, polyester, polyurethane, neoprene, spandex and/or nylon. Mixed fabrics are also conceivable. Other substances which appear suitable in relation to biocompatibility, comfort, stability, elasticity and costs are also conceivable.

Alternatively or additionally, the strap 20 can have at least in some regions a cushioning material. The cushioning material can be manufactured, for example, from foam, cotton wool, woven fabric, cotton, wool, rubber, neoprene, gel and/or other elastic and/or viscoelastic materials. The cushioning material can increase the wear comfort and/or make the wearing of a cannula more agreeable.

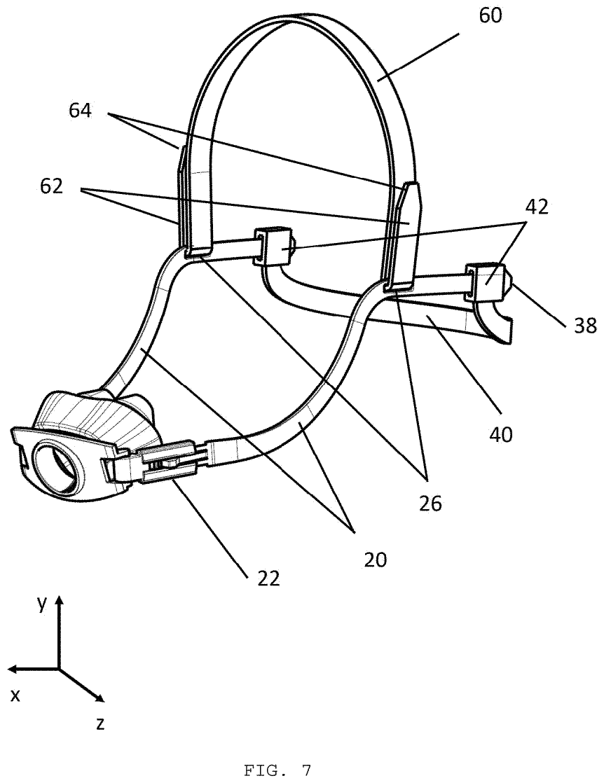

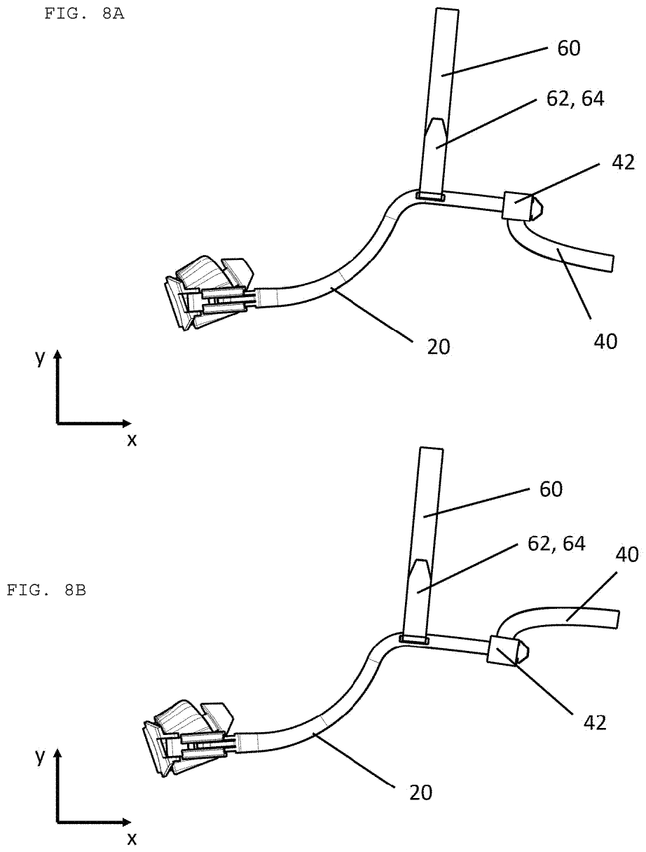

FIGS. 6 to 9 are views of the connecting element 10 according to the invention from FIG. 3, wherein FIG. 6A is a view of the connecting element 10 from above, FIG. 6B is a view from above of the connecting element 10 in a use state on the head 95 of a user, FIG. 7 is a perspective view of the connecting element 10, FIG. 8 is a view of the connecting element 10 from the side, and FIG. 9 is a view of the connecting element 10 from the front.

The straps 20 can be straight and/or bent or curved in the x/z coordinate system. Preferably, there is at least one curve and/or bend of the strap 20 within the x/z coordinate system. As a result of the bend or curve of the straps 20 in the x/z coordinate system, the straps 20 can run from the patient interface 90 to the ears and can be adapted to the shape of the head of the user and/or patient. As a result of the bend or curve of the straps 20 in the x/z coordinate system, the connecting element 10 can be made narrower at the patient interface 90 than in the regions which lie against the head 95 of the user in the use state (see FIG. 6B). As a result of the bend or curve in the z-direction, the end 38 of the strap lies above and/or below the beginning of the strap 20.

The portions 31, 33, 35, 37 can be straight and/or bent or curved in the x/z coordinate system. All the portions 31, 33, 35, 37 can be bent or curved in the x/z coordinate system. In some embodiments, it is also possible for only some or only one of the portions 31, 33, 35, 37 to be bent or curved in the x/z coordinate system.

In the concrete exemplary embodiment according to FIG. 6A-B, at least one portion of the strap 20 is bent or curved in the x/z coordinate system. By way of example, portion 31 is curved in the x/z coordinate system by an angle β. The inflection point 32 then lies above the inflection point 34 in the z-direction. The angle β is in a range from 120° to 180°. For example, the angle β is in a range between 130° and 170°.

The strap 20 is preferably bent or curved at least in part between the beginning 30 and the end 38 in two planes, namely both in the x/y coordinate system and in the x/z coordinate system.

The behind-the-ear element has a length L and a width B. In a simple exemplary embodiment, the length L can be equal to the width B. The ratio of the length L to the width B of the behind-the-ear element 40 can be about 1:1 (not shown). In preferred embodiments, the ratio of the length L to the width B of the behind-the-ear element 40 can preferably be about 5:1 or more.

It is apparent from FIG. 6 and FIG. 7 that the behind-the-ear element 40 in this exemplary embodiment is adapted and configured to be so long that it can run from the end 38 of one strap 20 to the end 38 of the second strap 20. In a use state, the behind-the-ear element 40 is arranged at the back of the head of the user. In the exemplary embodiment according to FIG. 3 ff, only a single behind-the-ear element 40 is arranged on the straps 20.

The behind-the-ear element 40 has, for example, an elongate form with two ends 44. The ends 44 can be shaped such that they form one or more retaining elements 42. The behind-the-ear element 40 can be coupled to the ends 38 of the two straps 20 by way of the, for example, two retaining elements 42. This coupling can be reversible or irreversible. This coupling can be adapted to be movable or unmovable. A length adjustment can take place by way of the coupling of the behind-the-ear element 40 and the strap 20. Depending on the position at which the retaining elements 42 are placed on the strap 20, the connecting element 10 can be adjusted to be wider or narrower (see also hereinbelow).

In the exemplary embodiment shown in FIGS. 3 and 5 to 8, the connecting element 10 can comprise, in addition to the strap 20 and the behind-the-ear element 40, a further element in the form of at least one head band 60.

The head band 60 can be fastened to the strap 20. The fastening can be adapted to be releasable and/or non-releasable. Preferably, the head band 60 can be releasably fastened to the strap 20. The head band 60 can pass through the apertures 26 of the straps 20 in order to couple the head band 60 to the straps 20.

The head band 60 can have an elongate narrow shape. The length L of the head band 60 is substantially greater than the width B of the head band 60. The ratio of the length L to the width B of the head band 60 can be in a range from about 10:1 to about 50:1. For example, the ratio of the length L to the width B of the head band 60 is at least about 10:1. In some embodiments, the ratio of the length L to the width B of the head band 60 is at least about 20:1. In a preferred embodiment, the ratio of the length L to the width B of the head band 60 is about 28:1 or more. The head band 60 preferably has an elongate, narrow shape.

The width B of the head band 60 can be constant or varying. In one exemplary embodiment, the width B of the head band 60 can be constant. The width B of the head band 60 can be in a range from about 1 mm to about 50 mm, preferably in a range from about 3 to about 30 mm. Particularly preferably, the width B of the head band 60 is from about 5 to about 20 mm. In a concrete embodiment, the width B of the head band 60 is about 12 mm.

The length L of the head band 60 can be in a range from about 150 mm to about 500 mm. Preferably, the length L of the head band 60 is from about 300 mm to about 500 mm. In a concrete exemplary embodiment, the length L of the head band 60 is by way of example about 340 mm.

The head band 60 can be manufactured from a textile. The material of the head band 60 is preferably soft and flexible. The head band 60 is particularly preferably configured to be stretchable. Conceivable textiles can be cotton, silk and/or synthetic fibers such as, for example, of polyester, polyurethane, neoprene, spandex and/or nylon. Mixed fabrics are also conceivable. Other substances which appear suitable in relation to biocompatibility, comfort, stability, elasticity and costs are also conceivable.

In a simple embodiment, the head band 60 is manufactured from only one textile and is adapted and configured to impart to the connecting element 10 additional hold and stability. Alternatively or additionally, the head band 60 can also have reinforcing and/or cushioning materials. This can be advantageous in order to increase the wear comfort and/or to facilitate fitting to the head 95. For example, it is conceivable that the head band 60 contains reinforcing structures, which provides the head band 60 in a shape that is optimal for the head shape.

The head band 60 can have a narrow, elongate shape with two ends 64 and two end regions 63. The two end regions 63 can account for up to about 50% of the total length L of the head band 60 as a whole. In preferred embodiments, the end regions 63 together account for about 25% of the total length L of the head band 60 as a whole. Fastening elements 62 can be arranged at one and/or at both end regions 63 of the head band 60.

In some embodiments, both end regions 63 of the head band 60 can comprise one or more fastening elements 62. For example, the end regions 63 of the head band 60 can comprise one or more Velcro fasteners and/or hooks and eyelets and/or buttons and buttonholes and/or snap fasteners and/or buckles or similar elements. With these fastening elements 62, the head band 60 can be reversibly fastened to the one or more straps 20. The fastening elements 62 are adapted and configured such that a length adjustment of the head band is possible.

The end regions 63 of the head band 60 preferably have one or more Velcro fasteners. For this purpose, there can be arranged at one or at both end regions 63 a Velcro region with flexible hooks, which can enter into a loadable, strong and reversible connection with the base material of the head band 60 (not shown).

For fastening the head band 60 to the straps, the two ends 64 and the end regions 63 of the head band 60 can pass through the apertures 26 of the straps 20. The head band 60 can then be bent in a loop shape so that the end regions 63 can be connected with other regions of the head band 60 (see for example FIG. 7). The connection can preferably be maintained by way of a Velcro fastener.

It is also conceivable that at least one of the two ends 64 and/or end regions 63 of the head band 60 is irreversibly connected to one of the straps 20 for protection against loss. Preferably at least one of the two end regions 63 of the head band 60 is fastened to the strap 20 in a length-adjustable manner as described above.

FIG. 10A-C shows a part-region of the strap 20 in different embodiments. It is apparent from FIG. 10 that the end portion 37 of the strap 20 can have different forms toward the end 38. In some embodiments, the end portion 37 can have a constant width B (FIG. 10A). In alternative embodiments, at least a part-region of the end portion 37 can have a particular form. For example, the end portion 37 can be widened at least in part relative to the fundamentally constant width B. An end portion 37 of widened form can be advantageous for the fastening of the behind-the-ear element 40 to the strap 20.

The end portion 37 can, for example, be widened at least in part to form an anchor element 25. The anchor element 25 can be configured such that the end 38 is widened in the shape of an arrow (FIG. 10B). The arrow-like widening can effectively prevent the behind-the-ear element 40 from unintentionally slipping off the strap 20. The anchor element 25 is not limited to the arrow-like shape. For example, the anchor element 25 can also be circular and/or oval and/or polygonal. The anchor element 25 can also have any other suitable geometry which allows the behind-the-ear element 40 to be coupled to the strap 20 and at the same time prevents unintentional slipping.

Alternatively or additionally, the end portion 37 of the strap 20 can comprise one or more fixing elements 24 which can positively influence the fastening of the behind-the-ear element 40 to the strap 20 (FIG. 10C). The fixing elements 24 on the strap 20 can be in the form of buttons, mushroom buttons, hooks, bars, studs, Velcro tape with hooks and/or loops or the like. One or more counterpart elements, that is to say buttonholes, holes, eyelets, loops, Velcro tape with hooks and/or loops or the like, can correspondingly be located on and/or at the ends 44 or end regions 43 of the behind-the-ear element 40 (not shown).

The fixing elements 24 can also be formed on the straps 20 by one or more thickened portions and/or indentations or similar structures, which permit latching on the strap 20 (not shown).

The fastening of one or more fixing elements 24 to a corresponding counterpart element can enhance and/or secure the fastening of the behind-the-ear element 40 to the straps 20. By arranging a plurality of fixing elements 24 on the strap, which are located at different positions of the strap 20, a further length adjustment of the connecting element 10 can be made possible.

The arrangement of additional fixing elements 24 on the end portion 37 of the strap 20 can be advantageous in particular in the case of positive pressure ventilation, because in this case a high force acts on the connecting element 10. Additional fixing elements 24 can secure and stabilize the fastening of the behind-the-ear element 40 to the straps 20.

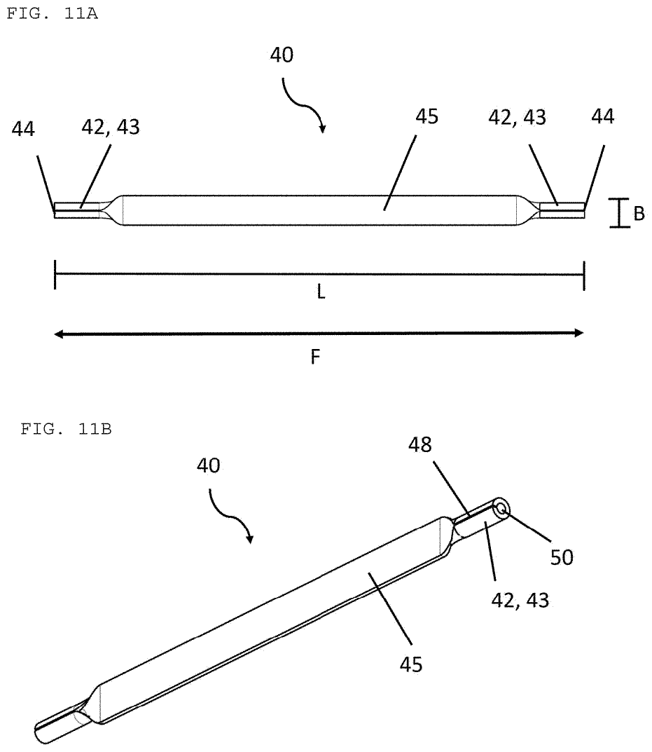

FIGS. 11 to 14 show different embodiments of the behind-the-ear element 40 of the connecting element 10 according to the invention from FIG. 3.

In the embodiment according to FIG. 3 ff, a single behind-the-ear element 40 is provided. The single behind-the-ear element 40 can be manufactured from one or more manufactured parts. In this concrete embodiment, the behind-the-ear element 40 comprises at least two end regions 43. At least one center piece 45 can be formed between the two end regions 43.

The two end regions 43 can account for up to about 100% of the total length L of the behind-the-ear element 40 as a whole, so that the behind-the-ear element 40 is formed without the center piece 45 (not shown). In alternative embodiments, a center piece 45, which can be of different lengths, is formed between the two end regions 43. For example, the two end regions 43 together can account for up to about 50% of the total length L of the behind-the-ear element 40 as a whole, and the center piece 45 can account for the remaining 50%. The center piece 45 can also be shorter or longer.

In preferred embodiments, the end regions 43 account for at most about 25% or less of the total length L of the behind-the-ear element 40 as a whole. The behind-the-ear element 40 can be coupled by way of its two end regions 43 to the end portions 37 of the two straps 20.

The center piece 45 can preferably account for at least about 50% of the total length L of the behind-the-ear element 40 as a whole. In preferred embodiments, the center piece 45 accounts for at least about 75% or more of the total length L of the behind-the-ear element 40 as a whole.

In the exemplary embodiments according to FIGS. 11 to 14, the length L of the behind-the-ear element 40 is substantially greater than the width B of the behind-the-ear element 40. For example, the ratio of the length L to the width B of the behind-the-ear element 40 is at least about 5:1. In some embodiments, the ratio of the length L to the width B of the behind-the-ear element 40 is at least about 20:1. In a preferred embodiment, the ratio of the length L to the width B of the behind-the-ear element 40 is about 30:1 or more. In the exemplary embodiments according to FIGS. 11 to 14, the behind-the-ear element 40 preferably has an elongate, narrow shape.

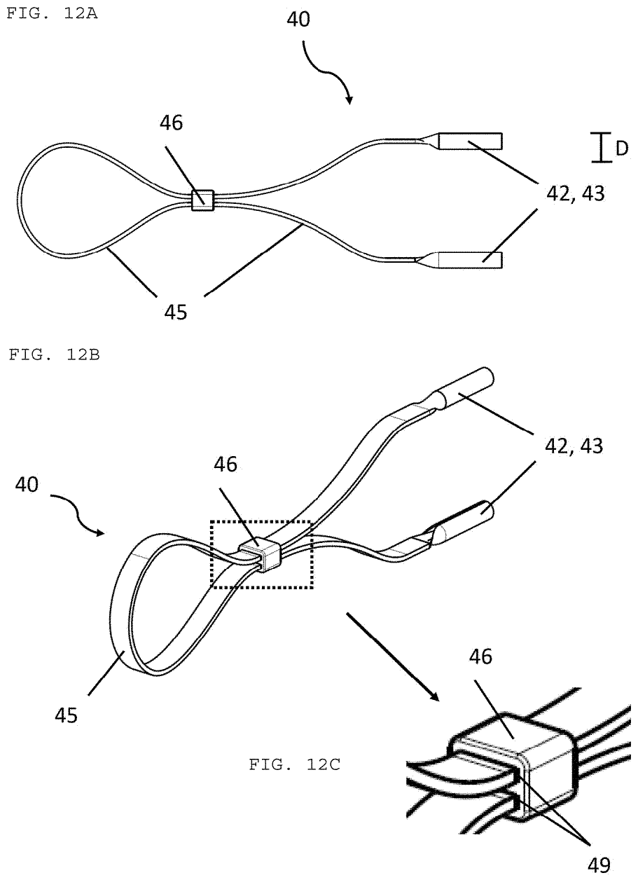

The width B of the behind-the-ear element 40 can be constant or varying. In one exemplary embodiment, the width B of the behind-the-ear element 40 can be substantially constant (FIGS. 11, 13, 14). In some embodiments, the width B of the behind-the-ear element 40 can also be varying (FIG. 12). The width B of the behind-the-ear element 40 can be in a range from about 0.2 mm to about 30 mm. Preferably, the width B of the behind-the-ear element 40 is from about 0.5 mm to about 25 mm, particularly preferably from about 7 mm and about 25 mm.

The length L of the behind-the-ear element 40 can be in a range from about 50 mm to about 400 mm. Preferably, the length L of the behind-the-ear element 40 is from about 100 to about 300 mm. In a concrete exemplary embodiment, the length L of the behind-the-ear element 40 is by way of example about 210 mm.

Preferably, the behind-the-ear element 40 is manufactured from an elastic material. The behind-the-ear element 40 can be manufactured from at least one material selected from silicones, thermoplastic elastomers (TPE), neoprenes or textiles. Conceivable textiles for the behind-the-ear element can be cotton, silk and/or synthetic fibers such as, for example, polyester, polyurethane, neoprene, spandex, nylon. The behind-the-ear element 40 is preferably manufactured from silicone.

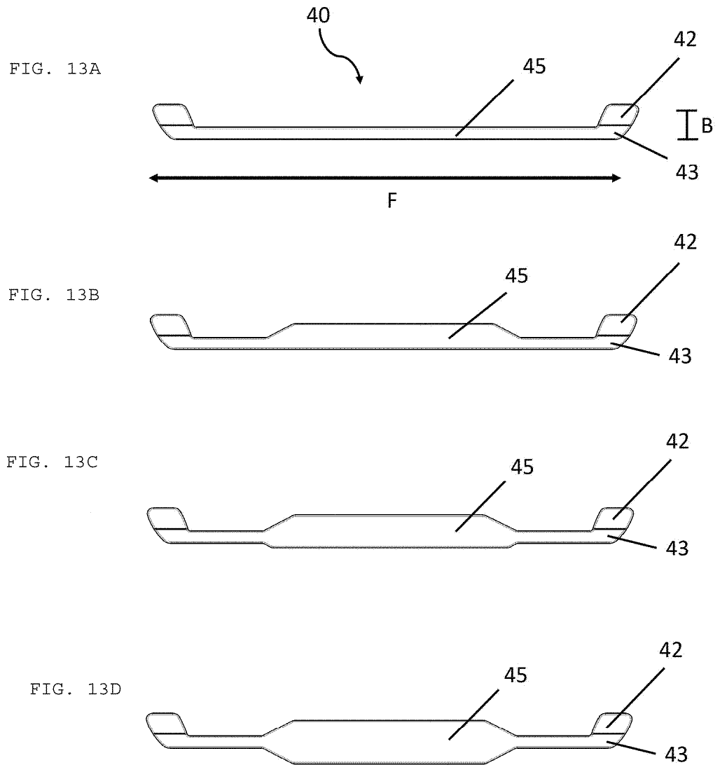

The behind-the-ear element 40 can be coupled to the straps 20. The behind-the-ear element 40 can comprise at least one, preferably two retaining elements 42. The retaining elements 42 can be formed within the end regions 43 and/or can be formed by the end regions 43 (see FIGS. 11, 12, 14). In alternative embodiments, the retaining elements 42 can also be molded on the end regions 43 (see FIG. 13). By way of the retaining elements 42, the behind-the-ear element 40 can be coupled to the straps 20. The retaining elements 42 can preferably be coupled to the end portions 37 of the straps 20.

The coupling of the behind-the-ear element 40 to the straps 20 can be irreversible or reversible. In some embodiments, it is conceivable that one retaining element 42 is coupled irreversibly to one strap 20 and the other retaining element 42 is coupled reversibly to the second strap 20 (not shown). It is also conceivable that the behind-the-ear elements 40 are integral components of the straps 20 and/or that straps 20 and behind-the-ear elements 40 are manufactured in one piece.

The coupling of the behind-the-ear element 40 to the straps 20 is preferably reversible. The coupling of the behind-the-ear element 40 to the straps 20 can be movable or unmovable. Preferably, the coupling of the behind-the-ear element 40 to the straps 20 is movable. By means of a movable coupling of the behind-the-ear element 40 to the straps 20, the positioning of the behind-the-ear element 40 within the connecting element 10 can be changed (see FIG. 8A and FIG. 8B).

FIG. 11 shows a simple exemplary embodiment of the behind-the-ear element 40 of the connecting element 10 according to the invention from FIG. 3, wherein FIG. 11A is a view from the side and FIG. 11 B is a perspective view.

In the concrete exemplary embodiment according to FIG. 11, the length L of the center piece 45 can account for up to about 90% and the length L of the two end regions 43 together can account for about 10% of the total length L of the behind-the-ear element 40. In the exemplary embodiment according to FIG. 11, the width B of the behind-the-ear element 40 can be substantially constant. The width B of the center piece 45 can be constant.

The end regions 43 can each be configured at least in part as retaining elements 42 in the form of elongate hollow bodies. The elongate hollow bodies can have, for example, a round and/or oval or angular cross-section. The elongate hollow bodies can be formed by joining each of the end regions 43 at least in part along the length L to form a closed profile. Joining of the end regions 43 to form a hollow body can be carried out by stitching, welding, adhesive bonding or the like, so that the end region 43 can have a joining groove 48. Joining of the end region 43 to form a hollow body is generally and preferably irreversible.

The retaining elements 42 so formed can be fitted onto the end portions 37 of the straps 20 (not shown). The end portions 37 of the straps 20 are then located at least in part in the lumen 50 of the retaining elements 42. The retaining elements 42 there hold the behind-the-ear element 40 on the end portions 37 of the straps 20 under pretension by friction.

In order that the connecting element 10 is held firmly on the head 95 of a user, the connecting element 10 must be adjusted to be slightly smaller than the circumference of the head 95 is large. Thus, in a use state, a tensile force F can act on the behind-the-ear element 40. The tensile force F generally runs in the direction of the length L of the behind-the-ear element 40. In the exemplary embodiment according to FIG. 11, the retaining elements 42 are in a plane with the center piece 45 along the tensile force F.

Holding can alternatively or additionally be implemented and/or assisted by fixing elements 24 and/or anchor elements 25 at the end portions 37 of the straps (see FIG. 10). For this purpose, at least one end region 43 of the behind-the-ear element 40 can have at least one buttonhole or hole or an eyelet or a loop or a Velcro tape with hooks and/or loops or the like. In some embodiments, it is also conceivable that the end region 43 has a plurality of buttonholes or holes or eyelets or loops or the like, so that a length adjustment is possible (not shown).

An additional fixing offers the advantage that the behind-the-ear element 40 can be firmly connected to the strap 20 and unintentional slipping on the strap 20 can be prevented. This is advantageous in particular in the case of the use of respiratory masks 90 for positive pressure ventilation, because the tensile force F for sealing the respiratory mask 90 can thus be enhanced by way of the behind-the-ear element 40.

FIG. 12 shows a further exemplary embodiment of the behind-the-ear element 40 of the connecting element 10 according to the invention from FIG. 3. The behind-the-ear element 40 shown in FIG. 12 corresponds substantially to the behind-the-ear element 40 shown in FIG. 11, but shows that the behind-the-ear element 40 can in some embodiments be substantially lengthened. The behind-the-ear element 40 can be up to about 200% longer than described above.

In this embodiment, the behind-the-ear element 40 can additionally comprise at least one eyelet 46. The eyelet 46 can have one or more openings 49 (see detail view of FIG. 12C). The behind-the-ear element 40 can pass through the one or more openings 49 such that the center piece 45 is formed at least in part into a loop. By displacing the eyelet 46, the length L of the behind-the-ear element 40 can be adjusted. The behind-the-ear element 40 is thus in itself adjustable in length. The eyelet 46 holds the behind-the-ear element 40 by friction and/or under pretension.

Alternatively or additionally, the eyelet 46 can contain a spring mechanism and/or a latching mechanism (not shown). The spring mechanism of the eyelet 46 can comprise a spring and an actuating element. By means of the spring mechanism, the behind-the-ear element 40 can be fixed in its position by clamping. The fixed position can be released by pressure on the actuating element, so as to permit adjustment of the position of the eyelet 46 and thus a length adjustment. The exemplary embodiment shown in FIG. 12 offers the advantage of a very flexible, simple, quick and reliable length adjustment of the behind-the-ear element 40.

FIG. 13A-D shows a further exemplary embodiment of the behind-the-ear element 40 of the connecting element 10 according to the invention from FIG. 3. FIG. 13 shows exemplary embodiments of the behind-the-ear element 40 in which the width B of the center piece 45 can be wider and/or narrower compared to the width B of the end regions 43. In these exemplary embodiments, the center piece 45 can account for about 50% of the total length L of the behind-the-ear element 40. In these exemplary embodiments, the end regions 43 together can account for about 50% of the total length L of the behind-the-ear element 40. The center piece 45 can also be shorter than shown.

FIG. 13A shows a simple embodiment of a behind-the-ear element 40 having a substantially constant width B. Only the retaining elements 42 are wider than the basic width B of the behind-the-ear element 40. FIGS. 13B-D show that the center piece 45 can be wider compared to the width B of the end regions 43. A wide center piece 45 can have a positive effect on the comfort and stability of the behind-the-ear element 40.

It is further apparent from FIG. 13 that the retaining elements 42 can be molded onto the end regions 43. The retaining elements 42 can be molded on above and/or below the end regions 43. The retaining elements 42 can also be configured such that they are arranged in a plane with the end regions 43 (see FIGS. 11, 12, 14).

In the embodiments according to FIG. 13, the retaining elements 42 are molded onto the end regions 43 such that the retaining elements 42 do not lie in a plane with the center piece 45 along the tensile force F. The tilting moment in the retaining element 42 effects stronger clamping and thus a better hold of the behind-the-ear element 40 on the strap 20.

A further advantage of these embodiments is that the position of the behind-the-ear element 40 in the use state can be varied. The behind-the-ear element 40 can be rotated so that, in a use state, it runs in the nape of the neck (not shown) (see FIG. 8A) or across the back of the head (not shown) (see FIG. 8B).

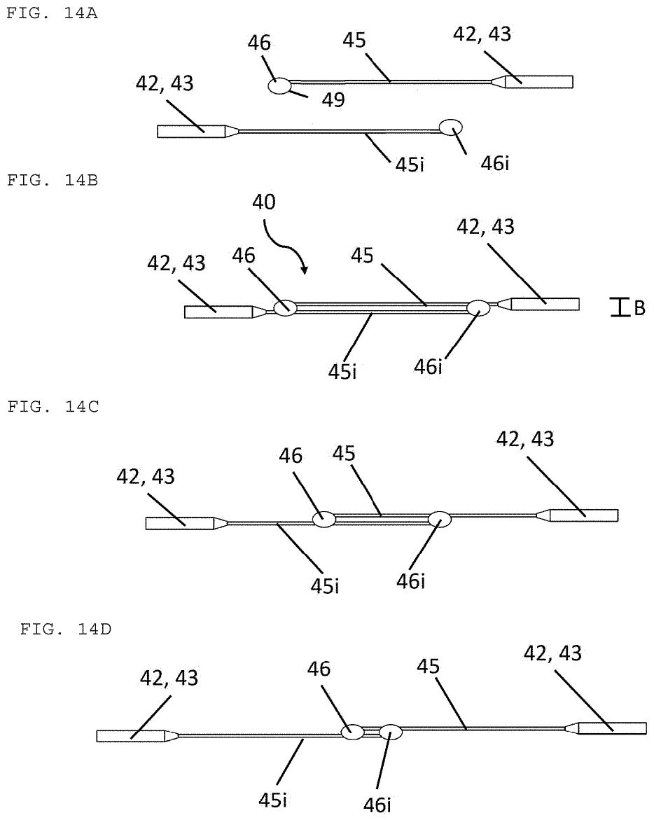

FIG. 14 shows an alternative exemplary embodiment of the behind-the-ear element 40 of the connecting element 10 according to the invention from FIG. 3.

The behind-the-ear element 40 according to FIG. 14A-D can be manufactured from a plurality of manufactured parts, for example two manufactured parts, which to produce a finished component can be joined together to form a behind-the-ear element 40. As a result of the two-part manufacture, a mechanism for length adjustment can be formed. The illustration in FIG. 14A shows the behind-the-ear element 40 in a partially disassembled state. FIGS. 14B-D show the behind-the-ear element 40 in an assembled, ready-for-use state.

FIG. 14A shows that the behind-the-ear element 40 can be manufactured from two manufactured parts of identical construction. Each of the two manufactured parts comprises an end region 43 with a retaining element 42, a center piece 45 and an eyelet 46. The eyelet 46 can in each case be fixedly connected to one end of the center piece 45. The end region 43 can be fixedly connected to the other end of the center piece 45. The eyelet 46 can have at least one opening 49. The opening 49 can be arranged in the eyelet 46 on one side. The opening 49 can be arranged in the eyelet 46 on both sides and form a through-passage through the eyelet 46.

The assembled behind-the-ear element 40 shown in FIGS. 14B-D shows that the behind-the-ear element 40 can in some embodiments comprise a length adjustment mechanism. For establishing the length adjustment mechanism, the behind-the-ear element 40 can comprise at least two or more eyelets 46, 46i and/or two or more center pieces 45, 45i.

For the concrete exemplary embodiment according to FIG. 14, the behind-the-ear element 40 preferably has two eyelets 46, 46i and two center pieces 45, 45i. The first eyelet 46 can be fixedly connected to the first center piece 45. The second center piece 45i can pass through the first eyelet 46 in a movable manner through the opening 49. The second eyelet 46i can be fixedly connected to the second center piece 45i. The first center piece 45 can pass through the second eyelet 46i in a movable manner through the opening 49. By means of such a two-part embodiment, simple and reliable length adjustment of the behind-the-ear element 40 can be made possible. The behind-the-ear element 40 is thus in itself adjustable in length.

The eyelets 46, 46i hold the center piece 45, 45i passing through the opening 49 of the eyelet 46, 46i by friction and/or under pretension and/or by way of a spring mechanism. It is also conceivable that the center pieces 45, 45i have thickened portions which permit length adjustment by latching (not shown).

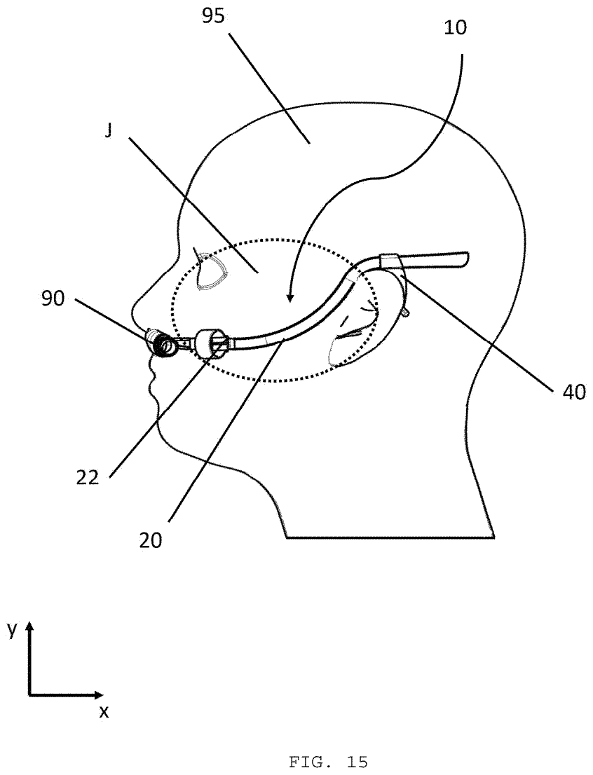

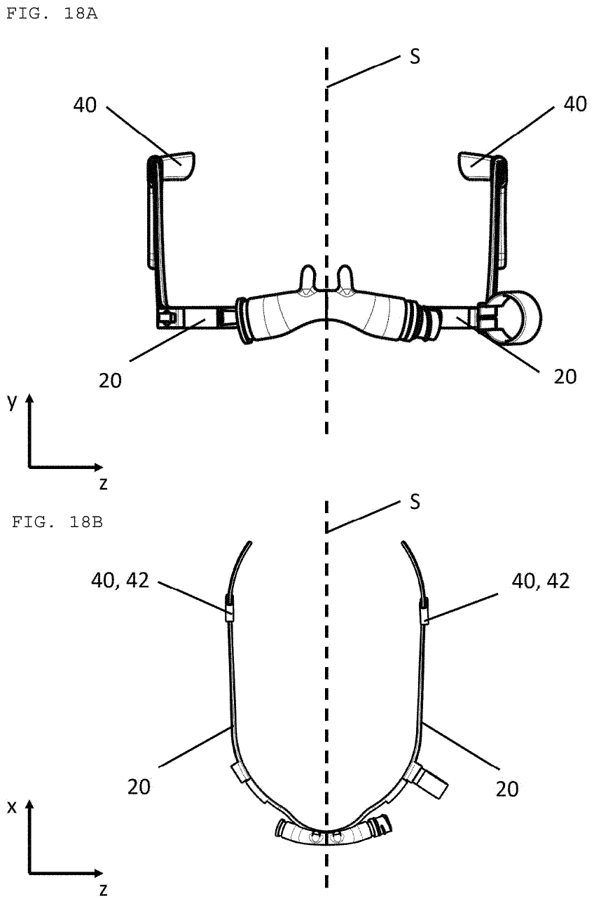

FIG. 15 is a schematic side view of an alternative embodiment of the connecting element 10 according to the invention in a use state on the head 95 of a user. FIGS. 16 to 18 are different views of the connecting element 10 according to the invention from FIG. 15, wherein FIG. 16 is a view of the connecting element 10 from the side, FIG. 17 is a perspective view of the connecting element 10, FIG. 18A is a view of the connecting element 10 from the front, and FIG. 18B is a view of the connecting element 10 from above.

In the alternative embodiment, the connecting element 10 comprises at least one strap 20 and at least one behind-the-ear element 40. Preferably, the connecting element 10 comprises two straps 20 and two behind-the-ear elements 40.

The connecting element 10 of the alternative embodiment according to FIG. 15 ff and the connecting element 10 according to FIG. 3 ff both comprise the straps 20 according to the invention which have been described in detail above. The straps 20 comprise the coupling system 22 for coupling to the patient interface 90 and the end portion 37 for coupling of the at least one behind-the-ear element 40. Coupling of the behind-the-ear element 40 to the straps 20 preferably takes place by way of the retaining elements 42. The retaining elements 42 can be configured and adapted to permit a length adjustment and/or to permit fixing and/or anchoring to the strap 20. A head band 60 is conceivable but not necessary in the alternative embodiment.

It is apparent from FIG. 15 that the connecting element 10 of the alternative embodiment, in a use state, is arranged, similarly to the connecting element 10 according to FIG. 3 ff, on the head 95 of a patient such that the strap 20 is arranged at least in part between the nose and the ear. The strap 20 is thereby located at least in part in the cheekbone region J. The strap 20 is configured according to the embodiment described in detail above.

The alternative embodiment according to FIG. 15 ff differs from the embodiments according to FIG. 3 ff in the form of the behind-the-ear element 40. In the embodiment according to FIG. 15 ff, at least two behind-the-ear elements 40 are provided, each of which can be coupled to one of the at least two straps 20.

The patient interface 90 can be in the form of a high-flow interface, HF interface for short. By way of the HF interface 90, the patient can receive high-flow oxygen therapy, HFOT for short. HFOT is a non-invasive form of respiratory support and/or respiratory therapy. In HFOT, oxygen and/or an oxygen-containing gas mixture can be supplied to the patient. The oxygen and/or the gas mixture can be administered at a very high gas flow rate of up to 60 l/min. The HF interface is usually in the form of a nasal cannula and can comprise nasal prongs 92. For example, the HF interface is in the form of a nasal cannula with at least one, preferably two nasal prongs 92. The nasal prongs 92 (see FIG. 16) can be inserted at least partially into the nose (not shown).

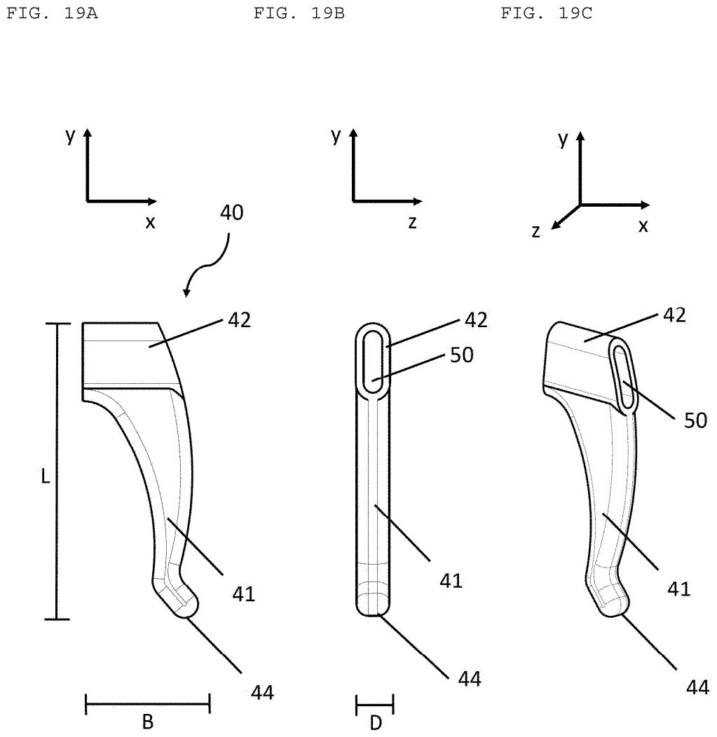

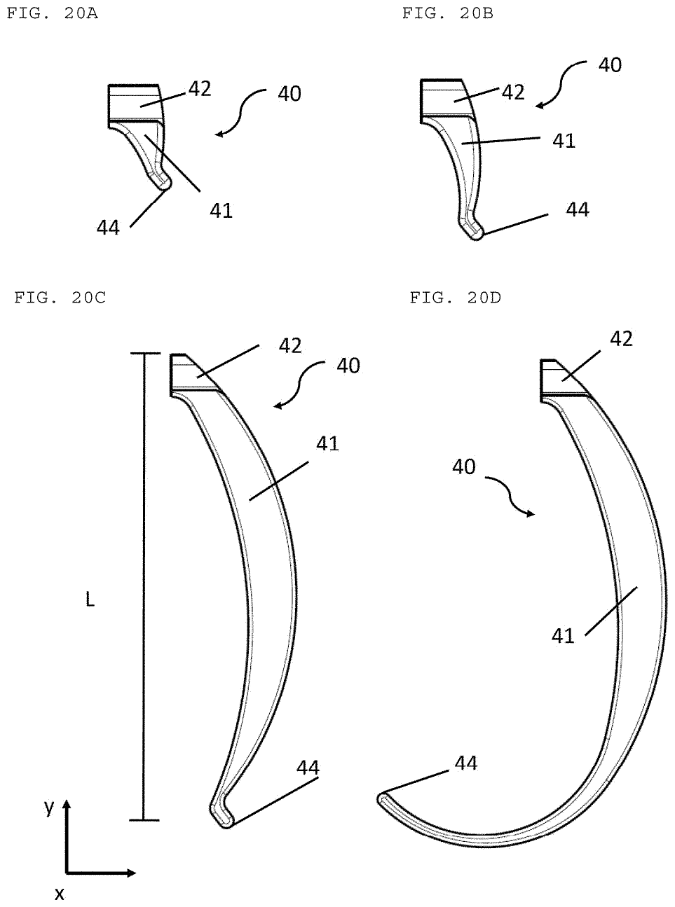

FIGS. 19 and 20 show exemplary embodiments of the behind-the-ear element 40 of the connecting element 10 according to the invention according to FIG. 15 ff. Unlike in the exemplary embodiments according to FIGS. 11 to 14, the behind-the-ear element 40 can be made shorter. In these exemplary embodiments, the ratio of the length L to the width B of the behind-the-ear element 40 can in each case be less than about 20:1, preferably less than about 10:1, particularly preferably less than about 5:1.

FIG. 19A-C shows a simple exemplary embodiment of the behind-the-ear element 40 of the connecting element 10 according to the invention according to FIG. 15, wherein FIG. 19A is a view from the side, FIG. 19B is a view from the front, and FIG. 19C is a perspective view.

In the embodiment according to FIG. 15 ff, a behind-the-ear element 40 is provided for each strap 20. Preferably, the connecting element 10 has two straps 20 each having a behind-the-ear element 40. FIG. 19 shows a behind-the-ear element 40 which is arranged behind the ear in a use state (see FIG. 15). It is apparent from FIG. 19 that the length L of the behind-the-ear element 40 can be slightly greater than the width B. For example, the ratio of the length L to the width B of the behind-the-ear element 40 is less than about 20:1, preferably less than about 10:1, particularly preferably less than about 5:1. The ratio of the length L to the width B of the behind-the-ear element 40 in this concrete embodiment is by way of example about 3:1.

The behind-the-ear element 40 in this concrete embodiment comprises a retaining element 42. The retaining element 42 is in the form of a hollow body and comprises at least one lumen 50. The hollow bodies and the lumen 50 can have, for example, a round and/or oval and/or angular cross-section. In some embodiments, the behind-the-ear element 40 can consist only of the retaining element 42 (not shown). In alternative embodiments, the behind-the-ear element 40 can additionally comprise an end piece 41 (FIGS. 19/20).

In order to establish a use state, the behind-the-ear element 40 can be coupled to the strap 20. Coupling takes place by way of the retaining element 42, which can be coupled to the end portion 37 of a strap 20. Preferably, a behind-the-ear element 40 is coupled to both straps 20. The end portion 37 of a strap 20 is then located at least in part in the lumen 50 of a retaining element 42. The retaining elements 42 there hold the behind-the-ear element 40 at the end portions 37 of the straps 20 under pretension by friction. Alternatively or additionally—as described hereinabove in relation to FIG. 10—holding can be implemented and/or assisted by fixing elements 24 and/or anchor elements 25 at the end portions 37 of the straps and with corresponding counterpart elements on the behind-the-ear elements 40.

Coupling of the behind-the-ear elements 40 to the straps 20 can allow the connecting element 10 to be adapted to the respective head shape. The positioning of the behind-the-ear elements 40 on the end portions 37 of the straps 20 can preferably be variably adjustable. For smaller head shapes, the behind-the-ear element 40 can be pushed on the strap 20 in the direction toward the beginning 30. For larger head shapes, the behind-the-ear element 40 can be pushed on the strap 20 in the direction toward the end 38. This offers the advantage of very simple and quick size adjustment of the connecting element 10.

In contrast to the behind-the-ear element 40 according to FIG. 3 ff, the behind-the-ear element 40 according to FIG. 15 ff does not run across the back of the head of a user in a use state. Instead, the behind-the-ear element 40 according to FIG. 15 ff is arranged on and/or behind the ear in a use state. The behind-the-ear element 40 can be substantially shorter than in the embodiments according to FIGS. 11 to 14. The length L of the behind-the-ear element 40 can be varied by center pieces 45 and/or end pieces 41 of different lengths.

The end piece 41 according to FIGS. 19/20 can be substantially shorter than the corresponding center piece 45 of the embodiments according to FIGS. 11-14. FIG. 19A-C shows a behind-the-ear element 40 which is arranged behind the ear in a use state (see FIG. 15). The length L of the end piece 41 can be in a range from about 10 mm to about 150 mm. Preferably, the length L of the end piece 41 is from about 15 mm to about 100 mm. In the concrete exemplary embodiment according to FIG. 19, the length L of the end piece 41 is by way of example about 32 mm.

FIG. 20A-D shows alternative exemplary embodiments of the behind-the-ear element 40 of the connecting element 10 according to the invention according to FIG. 15 from the side. It is apparent from FIG. 20 that the end piece 41 of the behind-the-ear element 40 can have different lengths L. FIG. 20A shows a particularly short exemplary embodiment of a behind-the-ear element 40 with a total length L of about 20 mm.

The behind-the-ear element 40 can have different lengths L (FIG. 20A-D). For example, the behind-the-ear element 40 can be long and bent such that it encloses the ear in a use state (not shown) such that the connecting element 10 is held particularly advantageously (FIG. 20D). FIG. 20D shows a particularly long exemplary embodiment of a behind-the-ear element 40 with a total length L of about 150 mm.

To sum up, the present invention provides the following items:
1. A connecting element for a patient interface, which connecting element comprises at least one strap which is configured for connection to the patient interface and comprises two straps and at least one behind-the-ear element.
2. The connecting element of item 1, wherein the at least one behind-the-ear element can be coupled reversibly to the two straps.
3. The connecting element of item 1, wherein the at least one behind-the-ear element can be coupled irreversibly to the two straps.
4. The connecting element of at least one of the preceding items, wherein the two straps comprise a beginning and an end, a coupling system for coupling a strap to the patient interface being disposed and configured adjacent to the beginning, and wherein an end portion to which the behind-the-ear element can be coupled is arranged adjacent to the end.
5. The connecting element of at least one of the preceding items, wherein the two straps are produced at least from a stiffening and at the same time flexible material and/or composite material selected from plastics materials, metals, silicones and foams, preferably from polypropylene.
6. The connecting element of at least one of the preceding items, wherein the two straps have a length L and a width B, a ratio of the length L to the width B being at least about 10:1, preferably at least about 20:1, particularly preferably at least about 25:1.
7. The connecting element of at least one of the preceding items, wherein the behind-the-ear element is manufactured from an elastic material selected from silicones, thermoplastic elastomers, neoprenes and textiles, preferably from silicone.
8. The connecting element of at least one of the preceding items, wherein the behind-the-ear element comprises at least one retaining element which can be coupled to an end portion of a strap.
9. The connecting element of item 8, wherein the retaining element is in the form of a hollow body with a lumen into which the end portion of the strap is placed for coupling at at least one position.
10. The connecting element of at least one of items 8 and 9, wherein the retaining element is displaceably coupled at any desired positions of the end portion and a length adjustment of the connecting element is thus provided.
11. The connecting element of at least one of items 8 to 10, wherein the end portion comprises at least one anchor element for anchoring the coupling of the retaining element to the straps.
12. The connecting element of at least one of items 8 to 11, wherein the end portions alternatively or additionally comprise one or more fixing elements for fixing the coupling of the behind-the-ear element to the straps.
13. The connecting element of item 12, wherein the fixing of the behind-the-ear element to the straps permits a stepwise length adjustment of the connecting element.
14. The connecting element of at least one of the preceding items, wherein the behind-the-ear element has a length L and a width B, a ratio of the length L to the width B of the behind-the-ear element being at least about 1:1, preferably at least about 5:1.
15. The connecting element of at least one of the preceding items, wherein the connecting element comprises two straps and one behind-the-ear element with two retaining elements, wherein one retaining element is coupled to one strap of the two straps and the second retaining element is coupled to the other strap of the two straps, and wherein the one behind-the-ear element runs across a back of a head of a user or patient in a use state.
16. The connecting element of at least one of the preceding items, wherein a ratio of a length L to a width B of the behind-the-ear element is at least about 5:1, preferably at least about 20:1, particularly preferably at least about 30:1.
17. The connecting element of at least one of the preceding items, wherein the behind-the-ear element is in itself adjustable in length and comprises at least one eyelet which is adapted and configured to permit a length adjustment.
18. The connecting element of at least one of the preceding items, wherein the connecting element comprises two straps and two behind-the-ear elements each with a retaining element, wherein a behind-the-ear element is coupled to each of the two straps, and wherein the behind-the-ear elements run behind a ear of a user or patient in a use state.
19. The connecting element of item 18, wherein a ratio of the length L to the width B of the behind-the-ear elements in each case is less than about 20:1, preferably less than about 10:1, particularly preferably less than about 5:1.
20. A system for ventilation and/or respiratory support, wherein the system comprises at least a patient interface, a connecting element for connection to the patient interface, a ventilator, and at least one line, the patient interface being coupled with the connecting element and being connected by the connecting element to a head of a patient, and the patient interface and the ventilator being connected together in a gas-conducting manner by the at least one line, and wherein the connecting element is adapted and configured as set forth in any of the preceding items.

Although the present invention has been described in detail with reference to the exemplary embodiments, it will be clear to the person skilled in the art that the invention is not limited to these exemplary embodiments. Rather, modifications are possible such that individual features are omitted or different combinations of the described individual features can be implemented, providing that there is no departure from the scope of protection of the accompanying claims. The present disclosure includes all combinations of the described individual features.

List of reference signs

| | |
|---|---|
| 10 | Connecting element |
| 20 | Strap |
| 22 | Coupling system |
| 24 | Fixing element |
| 25 | Anchor element |
| 26 | Coupling element |
| 30 | Beginning |
| 31 | Portion |
| 32 | Inflection point |
| 33 | Portion |
| 34 | Inflection point |
| 35 | Portion |
| 36 | Inflection point |
| 37 | End portion |
| 38 | End |
| 40 | Behind-the-ear element |
| 41 | End piece |
| 42 | Retaining element |
| 43 | End region |
| 44 | End |
| 45 | Center piece |
| 45i | Second center piece |
| 46 | Eyelet |
| 46i | Second eyelet |
| 48 | Connecting groove |
| 49 | Opening |
| 50 | Lumen |
| 60 | Head band |
| 62 | Fastening element |
| 63 | End region |
| 64 | End |
| 70 | Ventilator |
| 80 | Line |
| 90 | Patient interface |
| 91 | Nasal pillow |
| 92 | Nasal prong |
| 93 | Hose support |
| 95 | Head |
| 100 | System |
| B | Width |
| D | Thickness |
| F | Tensile force |
| J | Cheekbone region |
| L | Length |
| M1, M2 | Center |
| R1, R2 | Radius |
| S | Plane of symmetry |
| x | x-axis |
| y | y-axis |
| z | z-axis |
| α1, α2 | Central angle |
| β | Angle |

What is claimed is:

1. A connecting element for a patient interface, wherein the connecting element comprises at least one strap which is configured for connection to the patient interface and comprises two straps and at least one behind-the-ear element.

2. The connecting element of claim 1, wherein the at least one behind-the-ear element can be coupled reversibly to the two straps.

3. The connecting element of claim 1, wherein the at least one behind-the-ear element can be coupled irreversibly to the two straps.

4. The connecting element of claim 1, wherein the two straps comprise a beginning and an end, a coupling system for coupling a strap to the patient interface being disposed and configured adjacent to the beginning, and wherein an end portion to which the behind-the-ear element can be coupled is arranged adjacent to the end.

5. The connecting element of claim 1, wherein the two straps are produced at least from a stiffening and at the same time flexible material and/or composite material selected from plastics materials, metals, silicones and foams.

6. The connecting element of claim 1, wherein the two straps have a length L and a width B, a ratio of the length L to the width B being at least 10:1.

7. The connecting element of claim 1, wherein the behind-the-ear element is manufactured from an elastic material selected from silicones, thermoplastic elastomers, neoprenes and textiles.

8. The connecting element of claim 1, wherein the behind-the-ear element comprises at least one retaining element which can be coupled to an end portion of a strap.

9. The connecting element of claim 8, wherein the retaining element is in the form of a hollow body with a lumen into which the end portion of the strap is placed for coupling at at least one position.

10. The connecting element of claim 8, wherein the retaining element is displaceably coupled at any desired positions of the end portion and a length adjustment of the connecting element is thus provided.

11. The connecting element of claim 8, wherein the end portion comprises at least one anchor element for anchoring the coupling of the retaining element to the straps.

12. The connecting element of claim 8, wherein the end portion alternatively or additionally comprises one or more fixing elements for fixing a coupling of the behind-the-ear element to the straps.

13. The connecting element of claim 12, wherein the fixing of the behind-the-ear element to the straps permits a stepwise length adjustment of the connecting element.

14. The connecting element of claim 1, wherein the behind-the-ear element has a length L and a width B, a ratio of the length L to the width B of the behind-the-ear element being at least 1:1.

15. The connecting element of claim 1, wherein the connecting element comprises two straps and one behind-the-ear element with two retaining elements, wherein one retaining element is coupled to one strap of the two straps and the second retaining element is coupled to the other strap of the two straps, and wherein the one behind-the-ear element runs across a back of a head of a user or patient in a use state.

16. The connecting element of claim 15, wherein a ratio of a length L to a width B of the behind-the-ear element is at least 5:1.

17. The connecting element of claim 1, wherein the behind-the-ear element is in itself adjustable in length and comprises at least one eyelet which is adapted and configured to permit a length adjustment.

18. The connecting element of claim 1, wherein the connecting element comprises two straps and two behind-the-ear elements each with a retaining element, wherein a behind-the-ear element is coupled to each of the two straps, and wherein the behind-the-ear elements run behind a ear of a user or patient in a use state.

19. The connecting element of claim 18, wherein a ratio of a length L to a width B of the behind-the-ear elements in each case is less than 20:1.

20. A system for ventilation and/or respiratory support, wherein the system comprises at least a patient interface, a connecting element for connection to the patient interface, a ventilator, and at least one line, the patient interface being coupled with the connecting element and being connected by the connecting element to a head of a patient, and the patient interface and the ventilator being connected together in a gas-conducting manner by the at least one line, and wherein the connecting element is adapted and configured as set forth in claim 1.
